(19) United States
(12) Patent Application Publication
(10) Pub. No.: US 2026/0083310 A1
(43) Pub. Date: Mar. 26, 2026

(54) OPTICAL DETECTION OF MIDDLE EAR EFFUSION AND INFECTION

(71) Applicant: Northwestern University, Evanston, IL (US)

(72) Inventors: Claus-Peter Richter, Skokie, IL (US); Joaquin Cury, Chicago, IL (US); Jordan Michael Villa, Chicago, IL (US)

(21) Appl. No.: 19/335,124

(22) Filed: Sep. 22, 2025

Related U.S. Application Data
(60) Provisional application No. 63/696,977, filed on Sep. 20, 2024.

(51) Int. Cl. A61B 1/227; A61B 1/00; A61B 1/06

(52) U.S. Cl. CPC A61B 1/227; A61B 1/00055; A61B 1/0638

(57) ABSTRACT

An optical detection system for diagnosis of ear conditions includes a first light source that emits light of a first wavelength and a second light source that emits light of a second wavelength that differs from the first wavelength. An imaging device is positioned proximate to the first light source and the second light source. The imaging device is sized to fit within an ear canal of a patient to capture one or more images within an ear of the patient. A processor is in communication with the imaging device and configured to analyze the one or more images to identify a laser speckle pattern in the one or more images and determine a diagnosis for the patient based at least in part on the laser speckle pattern identified in the one or more images.

### CROSS-REFERENCE TO RELATED APPLICATION

The present application claims the priority benefit of U.S. Provisional Patent App. No. 63/696,977 filed on Sep. 20, 2024, the entire disclosure of which is incorporated by reference herein.

### REFERENCE TO GOVERNMENT RIGHTS

This invention was made with government support under grant number DC018666 awarded by the National Institutes of Health. The government has certain rights in the invention.

### BACKGROUND

Middle ear effusions (MEEs), combined with an infection (e.g., otitis media (OM), etc.), are common clinical conditions in children aged 2 to 6. The etiology of OM is multi-faceted. The condition typically results from viral and bacterial infections, resulting in severe ear pain and transient conductive hearing loss (cHL). MEEs that result from fluid and mucus accumulation can also occur without an infection. In some situations, MEE will present with a transient cHL, as seen in OM. MEEs without inflammation rarely show discomfort or pain, which can delay a diagnosis. A timely diagnosis of OM and MEEs is important to prevent structural damage in the middle ear cavity and, more importantly, to prevent any irreversible hearing loss.

### SUMMARY

An illustrative optical detection system for diagnosis of ear conditions includes a first light source that emits light of a first wavelength and a second light source that emits light of a second wavelength that differs from the first wavelength. An imaging device is positioned proximate to the first light source and the second light source. The imaging device is sized to fit within an ear canal of a patient to capture one or more images within an ear of the patient. A processor is in communication with the imaging device and configured to analyze the one or more images to identify a laser speckle pattern in the one or more images and determine a diagnosis for the patient based at least in part on the laser speckle pattern identified in the one or more images.

In an illustrative embodiment, the one or more images include a tympanic membrane of the patient, and the laser speckle pattern is on the tympanic membrane. In another embodiment, the first light source emits white light. In one embodiment, the second light source emits light within a low-mid portion of the visible spectrum of light such that the second wavelength of the light is between 450 nanometers and 700 nanometers. In another embodiment, the first light source comprises a light-emitting diode. In another embodiment, the first light source comprises a pair of light-emitting diodes. In another embodiment, the second light source comprises a laser diode.

In one embodiment, the diagnosis is that the patient has middle car effusion with otitis media present or that the patient has middle car effusion without otitis media present. In another embodiment, the diagnosis is that the patient has otitis media with middle car effusion present or that the patient has otitis media without middle car effusion present. In an illustrative embodiment, the processor analyzes the one or more image to determine a speckle contrast of the laser speckle pattern. In another embodiment, the processor is configured to generate an alert responsive to the diagnosis being indicative of otitis media or middle car effusion.

An illustrative method of diagnosing car conditions includes illuminating an car canal of a patient with a first light source that emits light of a first wavelength and a second light source that emits light of a second wavelength that differs from the first wavelength. The method also includes capturing, by an imaging device positioned proximate to the first light source and the second light source, one or more images within an car of the patient. The method also includes analyzing, by a processor in communication with the imaging device, the one or more images to identify a laser speckle pattern in the one or more images. The method further includes determining, by the processor, a diagnosis for the patient based at least in part on the laser speckle pattern identified in the one or more images.

In one embodiment, the first light source comprises a pair of light-emitting diodes. In another embodiment, the second light source comprises a laser diode. In one embodiment, the second wavelength of light emitted by the second light source is between 450 nanometers and 700 nanometers. In another embodiment, the diagnosis is that the patient has middle car effusion with otitis media present or that the patient has middle ear effusion without otitis media present. In another embodiment, the diagnosis is that the patient has otitis media with middle ear effusion present or that the patient has otitis media without middle ear effusion present. In an illustrative embodiment, analyzing the one or more images includes determining a speckle contrast of the laser speckle pattern. In another embodiment, the one or more images include a tympanic membrane of the patient, and the laser speckle pattern represents an amount of blood flow through the tympanic membrane. In another embodiment, the method includes generating, by the processor, an alert responsive to the diagnosis being indicative of otitis media or middle ear effusion.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts a custom-made microfluidic channel model filled with milk in accordance with an illustrative embodiment.

FIG. 2A depicts a state of zero active flow of the milk in the microchannels in accordance with an illustrative embodiment.

FIG. 2B depicts active flow of milk throughout the microchannels in accordance with an illustrative embodiment.

FIG. 3A is an initial prototype of an LSCI device (i.e., otoscopic probe) for middle ear effusion detection in accordance with an illustrative embodiment.

FIG. 3B depicts an otoscopic probe connected to a computing system and display in accordance with an illustrative embodiment.

FIG. 4A is an image of the TM using the micro-camera and the white LED of the prototype device in accordance with an illustrative embodiment.

FIG. 4B is an image of the TM that was obtained with the micro-camera and the laser source of the prototype device in accordance with an illustrative embodiment.

FIG. 5 is a block diagram of a diagnostic system for identifying otitis media and middle ear effusion in accordance with an illustrative embodiment.

FIG. 6 is a flow diagram illustrating various operations performed by a system to diagnose car conditions in accordance with an illustrative embodiment.

### DETAILED DESCRIPTION

Diagnosing otitis media (OM) and middle car effusion (MEE) can be difficult, even for a highly trained clinician. The diagnosis typically involves otoscopy, tympanometry, and the clinical history of the patient. MEEs are frequently not diagnosed quickly enough because they are often asymptomatic, and patients typically do not seek clinical advice or treatment without resounding symptoms. It is especially a challenge to diagnose MEEs in patients with certain genetic conditions like Down syndrome or other situations where individuals are prone to getting MEEs secondary to the abnormal middle car structures and stenotic eustachian tubes.

Optical methods for diagnosing OM have emerged in recent years. For example, optical coherence tomography alongside standard otoscopic exams has been employed to case diagnostics of OM/MEE. Another approach uses visible light to enhance visualization of the vascularity that becomes prominent on the tympanic membrane as a result of OM. While promising, these techniques are limited to the patient first having symptoms that cause significant pain and/or hearing loss (i.e., enough to cause the patient to come to the clinic for evaluation). Thus, applications to assist patients in understanding whether they need clinical evaluation would be useful, particularly in younger children who cannot sufficiently communicate.

The amount of vascularization of the tympanic membrane can act as an indicator of whether an individual has OM. One way to analyze relative amounts of vascularity is by using laser speckle contrast imaging (LSCI). The method involves the use of coherent light sources, such as laser diodes. Alternatively, a different type of light source may be used. Reflection of the laser light by a non-uniform surface, such as a tissue, forms interference patterns with bright spots called speckles. The speckle pattern changes if the light is reflected from moving structures or particles, such as red blood cells. Quantifying the changes in the speckle pattern is therefore an efficient and accurate method of measuring blood flow. This has been shown in coronary and cerebral vessels. This method can also be used to identify fluids behind the tympanic membrane and to determine the tympanic membrane's vascularization, both of which are indicators for OM.

Existing devices and techniques for diagnosing OM/MEE do not use LSCI to characterize the increased vasculature of the tympanic membrane as a result of OM. LSCI has yet to be used in applications for otolaryngology. As discussed herein, LSCI coupled with artificial intelligence models, such as machine learning systems like support vector machines (SVMs) or decision trees, can provide highly sensitive results compared to other methods. Additionally, all-optical methods of diagnosing OM or MEE have been coupled to a traditional otoscope. The proposed device is a smaller, consumer-friendly device that is easy to use without standard medical education. This allows for earlier detection of OM or MEE and less downstream ramifications that might result without treatment.

Described herein are methods and optical devices/systems for diagnosing otitis media and middle car effusions by characterizing laser speckle patterns on the tympanic membrane. The proposed methods and systems can be used on individuals of any age. In one embodiment, the system includes a miniaturized camera, an illumination source, a coherent light source, and a processor to acquire and analyze speckle contrast images of the tympanic membrane using artificial intelligence models, such as machine learning algorithms. This approach can accurately diagnose middle car fluid accumulation or infections. The device and the developed software can be integrated into a small, consumer-friendly, over-the-counter design. Such a device helps, in the case of auricular discomfort, in the decision-making process regarding whether a clinical evaluation by a health professional is required. With otitis media being one of the most common clinical diagnoses, the proposed system provides consumers with a simple way of better understanding if further help is needed, while limiting unnecessary physician visits and antibiotic use.

The proposed system aims to increase the awareness of asymptomatic MEEs and to aid in its diagnosis. The diagnosis is based on coherent light reflected from the tympanic membrane. From captured images, the speckle contrast K can be calculated. The speckle contrast K is calculated for small pixel neighborhoods as the ratio of the standard deviation (σ) of the light intensity among the pixels and the corresponding average intensity (<I>) of all pixels in each neighborhood, as represented by Equation 1 below:

$$K = σ / ( < I > ) \qquad Equation 1$$

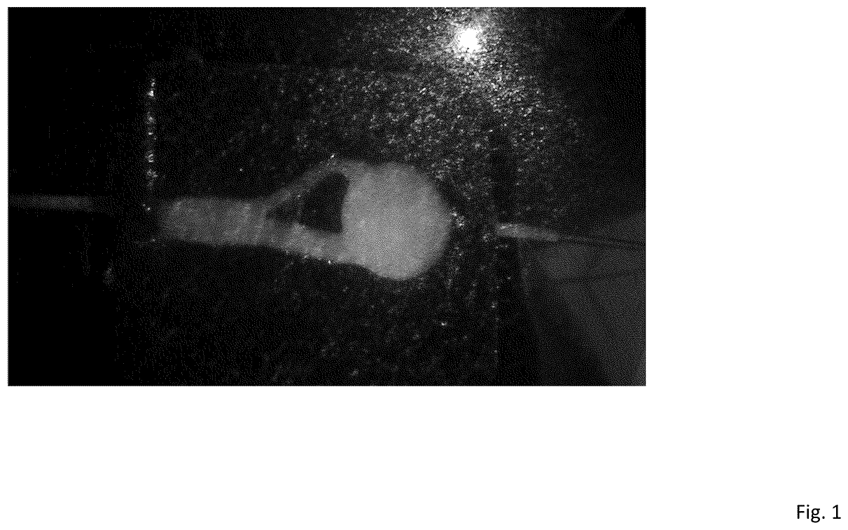

With the resulting data, machine learning (ML) models or other artificial intelligence models can be trained to decide whether normal or abnormal middle car conditions are found. The robustness of speckle contrast to determine fluid flow has been explored with a custom-made model. FIG. 1 depicts a custom-made microfluidic channel model filled with milk in accordance with an illustrative embodiment. The input of this channel was connected to a pump to control the flow rate. Additionally, a red laser was used to illuminate the microchannel, and with a microscope and a camera attached, the inventors obtained the speckle pattern under different flow rate conditions.

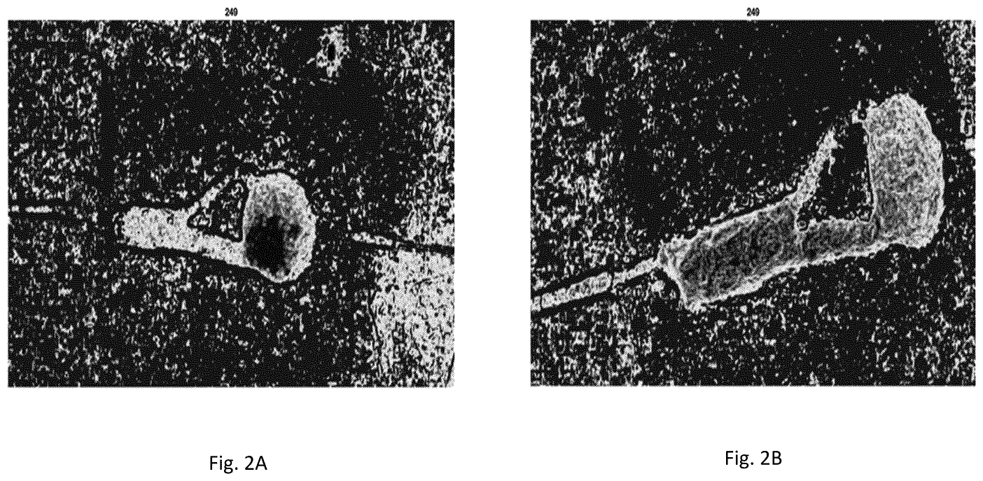

Using LSCI, flow versus no flow can be determined qualitatively. Relative flow rates can also be calculated using time correlation values. Examples are the relative heatmaps, as seen in FIG. 2, where FIG. 2A depicts a state of zero active flow of the milk in the microchannels in accordance with an illustrative embodiment. FIG. 2B depicts active flow of milk throughout the microchannels in accordance with an illustrative embodiment. The heat maps of FIG. 2 were obtained after running a custom program in MATLAB, although in alternative embodiments other software may be used.

In an illustrative embodiment, to form the device, two light sources are connected to a micro-camera (≈1 megapixel resolution) to capture high-quality images of the TM. The light sources can be a white light emitting diode (LED) and a laser diode emitting in the low-mid visible spectrum (e.g., wavelength range 495-570 nanometers (nm) or 630-670 nm). On one embodiment, the laser diode can emit light between 450 nm and 700 nm. In alternative embodiments, different types of light sources may be used and/or different wavelengths may be used. The wavelengths of the light emitted by the light sources are important as they determine light reflection and speckle size. In another illustrative embodiment, the device is small enough to be inserted into the external car canal easily.

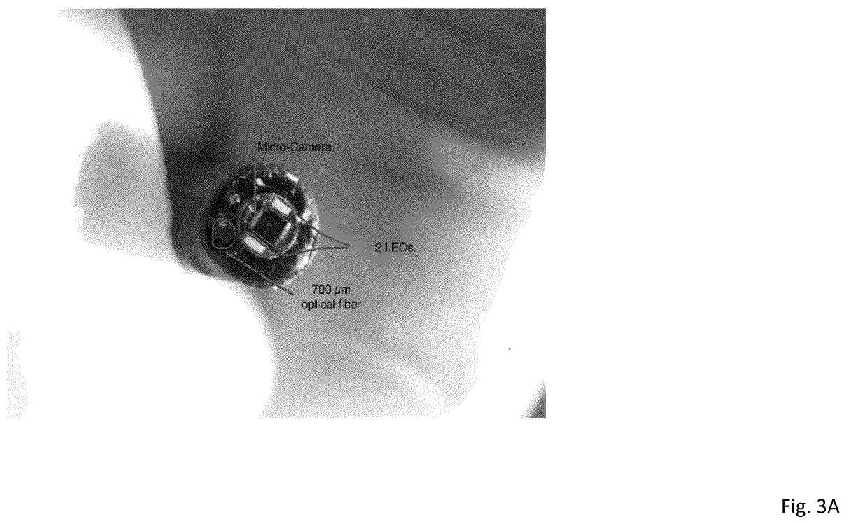

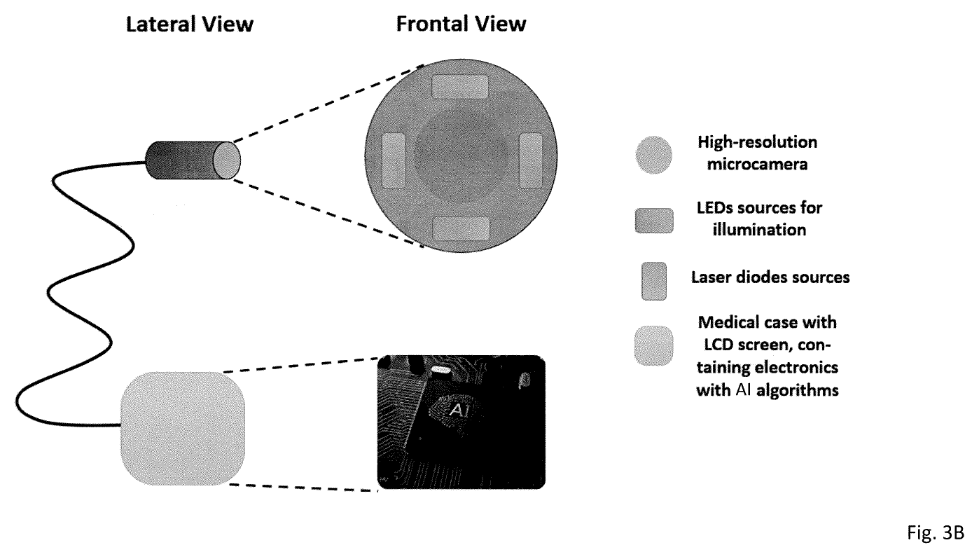

FIG. 3A is an initial prototype of an LSCI device (i.e., otoscopic probe) for middle car effusion detection in accordance with an illustrative embodiment. In FIG. 3A, the micro-camera, LEDs, and coherent light source (optical fiber) are labeled, and are fitted into a housing that is small enough to fit into the car canal of a patient. FIG. 3B depicts an otoscopic probe connected to a computing system and display in accordance with an illustrative embodiment. FIG. 3B includes both lateral and frontal views of the otoscopic probe, and depicts the high-resolution micro-camera, the LED light sources, the laser diode light sources, and the computing system with a display and electronics to perform artificial intelligence processing. In alternative embodiments, the device can include additional, fewer, and/or different components.

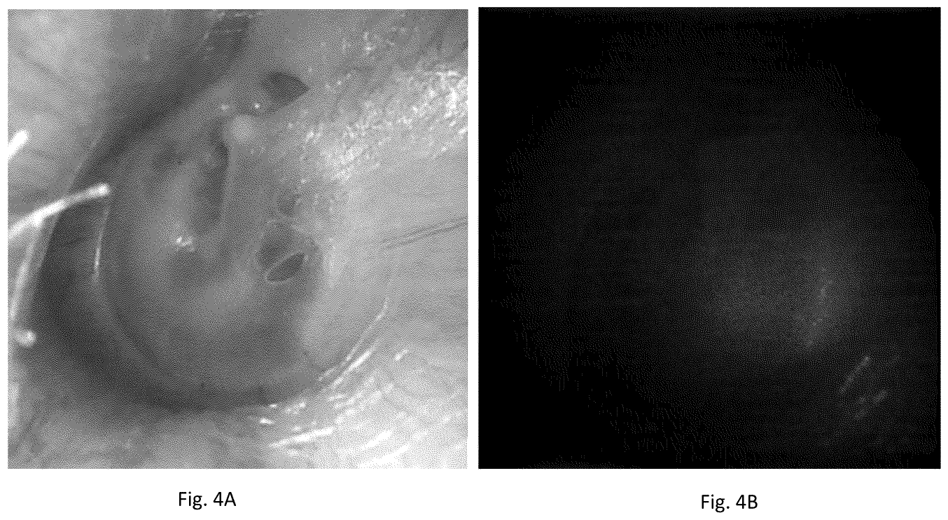

FIG. 4A is an image of the TM using the micro-camera and the white LED of the prototype device in accordance with an illustrative embodiment. This light source illuminates the car canal, so the placement of the optical system is optimal. FIG. 4B is an image of the TM that was obtained with the micro-camera and the laser source of the prototype device in accordance with an illustrative embodiment. The speckle pattern can be seen in the center of the plot.

In another illustrative embodiment, the image analysis can be performed by a processing apparatus (e.g., computing system) and can include a support vector machine (SVM), trained decision tree machine learning software, other artificial intelligence models, and/or other software/applications to analyze the captured images. The processing system can be connected to an output device (e.g., a display) that can interpret the result and display it to the consumer based on the images of the TM. In an illustrative embodiment, the trained software is able to characterize the speckle pattern as one of the following: i) MEE with no OM present, ii) MEE with OM present, iii) OM with no MEE present, or iv) no OM or MEE present. The proposed method of diagnosing OM and MEE can be used within, but is not limited to, traditional otoscopes or over-the-counter (OTC) devices for at-home detection.

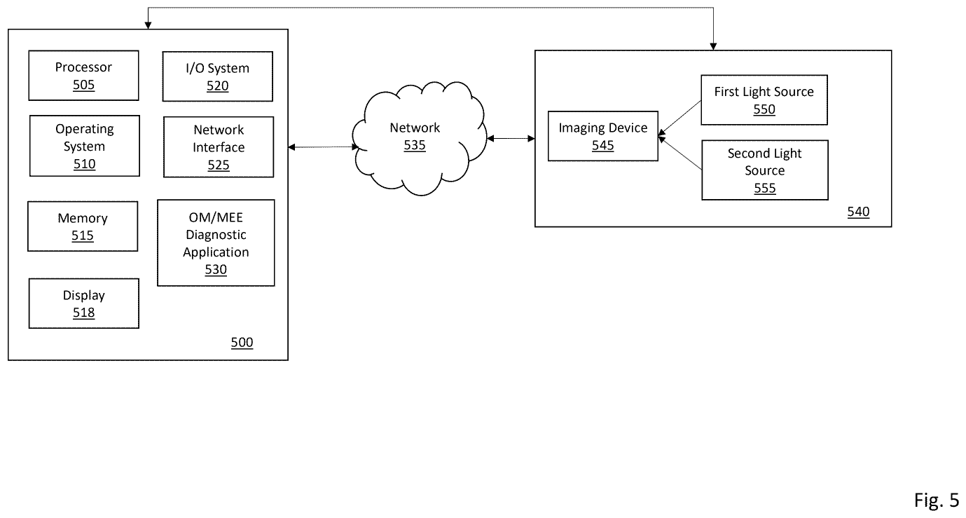

FIG. 5 is a block diagram of a diagnostic system for identifying otitis media and middle car effusion in accordance with an illustrative embodiment. The block diagram of FIG. 5 includes an imaging system 540 for capturing images of the car and a computing system 500 for processing captured images and making diagnoses, as described herein. In one embodiment, at least a portion of the computing system 500 can be remote from the imaging system 540, but in communication therewith through a network 535 or other form of wireless communication. In another embodiment, the computing system 500 can be incorporated into the imaging system 540.

As shown, the imaging system 540 includes an imaging device 545, which can be a micro-camera as described herein. The imaging device 545 also includes a first light source 550, which can be in the form of two light-emitting diodes (LEDs) (e.g., positioned on opposite sides of the imaging device 545). Alternatively, the first light source 550 can be a single LED. In another alternative embodiment, a different type of light source may be used as the first light source 550 and/or additional light sources may be used. A second light source 555 of the imaging system 540 can be a laser diode in one embodiment. In another embodiment, the second light source 555 can be in the form of a fiber optic cable that is positioned within a housing of the imaging device 545. In an illustrative embodiment, the first light source 550 emits white light and the second light source 555 emits light in the low-mid visible spectrum (e.g., wavelength range 495-570 nm or 630-670 nm). In alternative embodiments, the first light source 550 and/or the second light source 555 can emit light at other controlled wavelengths.

The computing system 500 includes a processor 505, an operating system 510, a memory 515, a display 518, an input/output (I/O) system 520, a network interface 525, and an OM/MEE diagnostic application 530. In alternative embodiments, the computing system 500 may include fewer, additional, and/or different components. The components of the computing system 500 communicate with one another via one or more buses or any other interconnect system. The computing system 500 can be any type of computing system (e.g., smartphone, tablet, laptop, desktop, etc.), including a dedicated standalone computing system that is designed to perform the OM/MEE analysis and diagnosis. As discussed, in one embodiment, at least a portion of the computing system 500 may be incorporated into the imaging system 540.

The processor 505 can be in electrical communication with and used to control any of the system components described herein. For example, the processor 505 can be used to execute the OM/MEE diagnostic application 530, control the imaging device 545, control the first light source 550, control the second light source 555, obtain and process captured image data, generate a diagnosis, generate an alert (e.g., a verbal or textual message to visit a physician as soon as possible) to the patient based on the diagnosis, etc. The processor 505 can be any type of computer processor known in the art and can include a plurality of processors and/or a plurality of processing cores. The processor 505 can include a controller, a microcontroller, an audio processor, a graphics processing unit, a hardware accelerator, a digital signal processor, etc. Additionally, the processor 505 may be implemented as a complex instruction set computer processor, a reduced instruction set computer processor, an x86 instruction set computer processor, etc. The processor 505 is used to run the operating system 510, which can be any type of operating system.

The operating system 510 is stored in the memory 515, which is also used to store programs, received image data, other patient data, OM/MEE data, network and communications data, peripheral component data, the OM/MEE diagnostic application 530, and other operating instructions. The memory 515 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 515 can be in the cloud to provide cloud storage for the system. Similarly, in one embodiment, any of the computing components described herein (e.g., the processor 505, etc.) can be implemented in the cloud such that the system can be run and controlled through cloud computing.

The I/O system 520 is the framework which enables users and peripheral devices to interact with the computing system 500. The display 518 can include a touch screen in some embodiments, and the touch screen can be part of the I/O system 520 that allows a user to make selections, control sub-systems, view results, etc. The display 518 can be any type of display, including a monitor, projector, LED screen, liquid crystal display (LCD) screen, etc., and can be used to present user interface screens, control screens, captured images, captured video, a diagnosis, and other data to the user. The I/O system 520 can also include one or more speakers, one or more microphones, a keyboard, a mouse, one or more buttons or other controls, etc. that allow the user to interact with and control the computing system 500 and/or the imaging system 540. The I/O system 520 also includes circuitry and a bus structure to interface with peripheral computing devices such as the imaging system 540, power sources, universal service bus (USB) devices, data acquisition cards, peripheral component interconnect express (PCIe) devices, serial advanced technology attachment (SATA) devices, high-definition multimedia interface (HDMI) devices, proprietary connection devices, etc.

The network interface 525 includes transceiver circuitry (e.g., a transmitter and a receiver) that allows the computing system 500 to transmit and receive data to/from other devices such as remote computing systems, servers, websites, the imaging system 540, etc. The network interface 525 enables communication through the network 535, which can be one or more communication networks. The network 535 can include a cable network, a fiber network, a cellular network, a wi-fi network, a landline telephone network, a microwave network, a satellite network, etc. The network interface 525 also includes circuitry to allow device-to-device communication such as Bluetooth® communication.

The OM/MEE diagnostic application 530 can include software and algorithms in the form of computer-readable instructions which, upon execution by the processor 505, performs any of the various operations described herein such as controlling the imaging system 540 to capture images, analyzing the captured images, identifying and analyzing speckle patterns, altering settings of the imaging system 540, analyzing patient data, determining an amount of vascularization present in an around the tympanic membrane, generating heat maps, making a diagnostic prediction based on the analysis, generating a warning if the analysis identifies a potential problem with the patient, displaying a warning or diagnosis, etc. The OM/MEE application 530 can utilize the processor 505 and/or the memory 515 and/or the display 518 as discussed above. In an alternative implementation, the OM/MEE diagnostic application 530 can be remote or independent from the computing device 500, but in communication therewith.

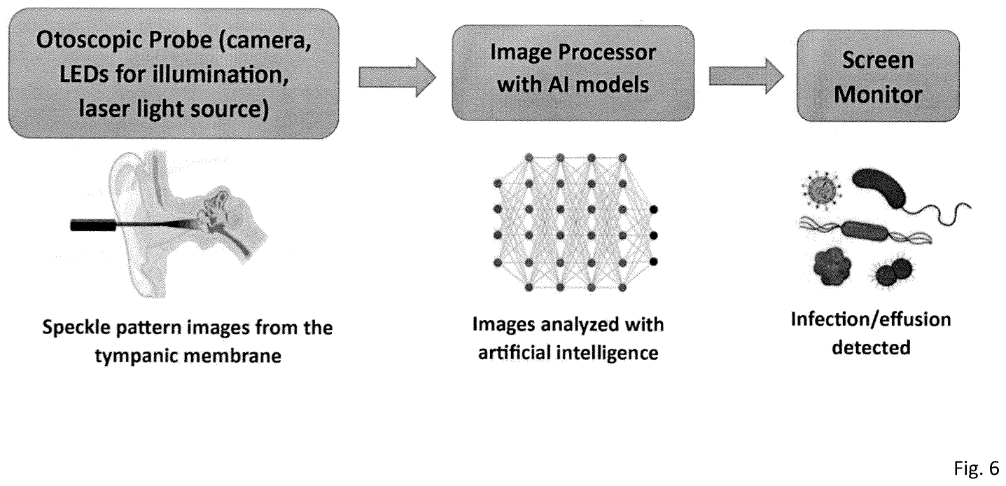

FIG. 6 is a flow diagram illustrating various operations performed by a system to diagnose ear conditions in accordance with an illustrative embodiment. In alternative embodiments, fewer, additional, and/or different operations may be performed. Also, the use of a flow diagram is not meant to be limiting with respect to the order of operations performed. As shown, an otoscopic probe device (e.g., the device of FIG. 3) that includes a camera that includes an optical sensor, one or more light sources to provide illumination for the camera's optical sensor, and one or more light sources (e.g., laser light sources) used to obtain speckle pattern images of the tympanic membrane of a patient. Specifically, low intensity laser light (e.g., below 5 milliwatts (mW)) can be used to obtain the speckle pattern images.

The obtained images are processed by a computing system using artificial intelligence, machine learning, etc. to identify car conditions such as OM or MEE. The analysis identifies vascularization and morphological changes that happen to the car during OM/EEE. Results of the analysis, including captured images, heat maps, alerts/warnings, recommendations, a diagnosis, etc. can be displayed on a screen monitor (i.e., display) to let the user know if infection and/or effusion has been detected. These results will allow for earlier detection to maintain optimal middle car health, while also helping individuals to avoid unnecessary healthcare visits.

While the proposed methods and systems can be used on individuals of any age and having any underlying conditions, they can be especially helpful for individuals with down syndrome. Down Syndrome (DS) is a genetic condition caused by the presence of an extra copy of chromosome 21, occurring in approximately 1 in every 775 newborns, with around 6,000 cases diagnosed each year in the United States. This genetic variation leads to anatomical phenotypes that increase the risk of health issues. Individuals with DS often experience intellectual disabilities, growth delays, and distinct facial features, along with a higher likelihood of complications such as gastrointestinal disorders, vision problems, congenital heart anomalies, and hearing loss.

Hearing loss, including both conductive and sensorineural types, is highly prevalent in children with DS, with estimates suggesting that 50-80% of them experience significant hearing loss, making it a critical health problem. This increased prevalence is due to anatomical factors such as narrow car canals, eustachian tube dysfunction, and frequent chronic otitis media and effusions (C-OME), which contribute to a greater risk of conductive hearing loss. Conductive hearing loss from conditions like C-OME has been further shown to have the potential to cause irreversible sensorineural hearing loss over time. Both conditions can significantly impact a child's speech and language development. Furthermore, evidence suggests that hearing loss is a significant modifiable risk factor for dementia, a known health disparity in people with DS, highlighting the critical need for early detection and effective management. While methods exist to identity middle car pathologies, traditional techniques make it difficult for medical providers to obtain an accurate diagnosis. The difficulty for clinicians often comes from distinguishing between serous middle car effusions and active infections, as both conditions can present with a bulging tympanic membrane and no other visible changes during otoscopy. Differentiating between the two is further complicated because active infections often have mild, non-specific symptoms. Additionally, examining children can be challenging, as they may be uncooperative or have difficulty staying still. As a result, diagnosis relies heavily on clinical experience, leading to a high rate of misdiagnosis. For example, the rate of misdiagnosis is up to 50% for acute otitis media cases diagnosed by pediatricians and up to 27% for cases diagnosed by otolaryngologists.

Traditional methods of diagnosing otitis media and effusions have progressed within the clinical setting. However, there is currently a lack of adoptable, affordable, accessible, and comfortable tools and resources for the average person to better understand these issues when present. Education and management of children with DS on the parent level is rudimentary and there is a notable lack of devices that are able to provide individuals or caretakers of DS patients with reliable information for seeking professional help in primary care centers. The proposed otoscope device helps to solve these problems by using laser speckle contrast imaging (LSCI) to highlight vascular and morphological characteristics of the tympanic membrane in healthy and pathologic conditions. This technique, coupled with advanced processing (e.g., support vector machines, deep learning, etc.) can provide non-trained individuals with information on when to seek professional medical advice to address middle ear effusions and infections at an early stage. The device improves diagnostic timing and accuracy of middle ear pathologies in individuals with DS, and in the general population.

Thus, described herein are methods and systems that utilize laser speckle contrast imaging to measure to detect MEE and OM, which is a more accurate and sensitive measurement technique than what is currently used in traditional systems. The proposed system can use trained machine learning or other artificial intelligence systems to provide quick and accurate diagnosis of OM and MEE. Additionally, the laser speckle contrast imaging can potentially be used to differentiate between serious and exudative middle car fluids. At-home designs of the proposed device can provide consumers with the knowledge needed to pursue further clinical evaluations in a cost-effective manner. At-home testing enhances patient comfort to increase compliance and provides accurate test results, particularly in children.

The proposed device therefore increases the efficiency and accuracy of detection of middle car pathology within at-home applications. This can lead to fewer cases of chronic hearing loss, the spread of pathology to the inner ear, and otosclerosis. Additionally, early detection (e.g., in children who cannot communicate ear discomfort properly) can help to prevent speech and language developmental delays.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. An optical detection system for diagnosis of ear conditions, the system comprising:
a first light source that emits light of a first wavelength;
a second light source that emits light of a second wavelength that differs from the first wavelength;
an imaging device positioned proximate to the first light source and the second light source, wherein the imaging device is sized to fit within an ear canal of a patient to capture one or more images within an ear of the patient; and
a processor in communication with the imaging device, wherein the processor is configured to:
analyze the one or more images to identify a laser speckle pattern in the one or more images; and
determine a diagnosis for the patient based at least in part on the laser speckle pattern identified in the one or more images.

2. The system of claim 1, wherein the one or more images include a tympanic membrane of the patient, and wherein the laser speckle pattern is on the tympanic membrane.

3. The system of claim 1, wherein the first light source emits white light.

4. The system of claim 1, wherein the second light source emits light within a low-mid portion of the visible spectrum of light such that the second wavelength of the light is between 450 nanometers and 700 nanometers.

5. The system of claim 1, wherein the first light source comprises a light-emitting diode.

6. The system of claim 1, wherein the first light source comprises a pair of light-emitting diodes.

7. The system of claim 1, wherein the second light source comprises a laser diode.

8. The system of claim 1, wherein the diagnosis is that the patient has middle ear effusion with otitis media present or that the patient has middle ear effusion without otitis media present.

9. The system of claim 1, wherein the diagnosis is that the patient has otitis media with middle ear effusion present or that the patient has otitis media without middle ear effusion present.

10. The system of claim 1, wherein the processor analyzes the one or more image to determine a speckle contrast of the laser speckle pattern.

11. The system of claim 1, wherein the processor is configured to generate an alert responsive to the diagnosis being indicative of otitis media or middle ear effusion.

12. A method of diagnosing ear conditions, the method comprising:
illuminating an ear canal of a patient with a first light source that emits light of a first wavelength and a second light source that emits light of a second wavelength that differs from the first wavelength;
capturing, by an imaging device positioned proximate to the first light source and the second light source, one or more images within an ear of the patient; and
analyzing, by a processor in communication with the imaging device, the one or more images to identify a laser speckle pattern in the one or more images; and
determining, by the processor, a diagnosis for the patient based at least in part on the laser speckle pattern identified in the one or more images.

13. The method of claim 12, wherein the first light source comprises a pair of light-emitting diodes.

14. The method of claim 12, wherein the second light source comprises a laser diode.

15. The method of claim 14, wherein the second wavelength of light emitted by the second light source is between 450 nanometers and 700 nanometers.

16. The method of claim 12, wherein the diagnosis is that the patient has middle ear effusion with otitis media present or that the patient has middle ear effusion without otitis media present.

17. The method of claim 12, wherein the diagnosis is that the patient has otitis media with middle ear effusion present or that the patient has otitis media without middle ear effusion present.

18. The method of claim 12, wherein analyzing the one or more images includes determining a speckle contrast of the laser speckle pattern.

19. The method of claim 12, wherein the one or more images include a tympanic membrane of the patient, and wherein the laser speckle pattern represents an amount of blood flow through the tympanic membrane.

20. The method of claim 12, further comprising generating, by the processor, an alert responsive to the diagnosis being indicative of otitis media or middle ear effusion.
